(19) United States
(12) Patent Application Publication
(10) Pub. No.: US 2016/0029968 A1
(43) Pub. Date: Feb. 4, 2016

(54) TRACKING SLOW VARYING FREQUENCY IN A NOISY ENVIRONMENT AND APPLICATIONS IN HEALTHCARE

(71) Applicant: ANALOG DEVICES, INC., Norwood, MA (US)

(72) Inventors: BORIS LERNER, SHARON, MA (US); GUOLIN PAN, WESTWOOD, MA (US); JIANG WU, WESTWOOD, MA (US); SHRENIK DELIWALA, ANDOVER, MA (US)

(73) Assignee: ANALOG DEVICES, INC., Norwood, MA (US)

(21) Appl. No.: 14/450,433

(22) Filed: Aug. 4, 2014

(51) Int. Cl. A61B 5/00; A61B 5/11; A61B 5/0205

(52) U.S. Cl. CPC A61B 5/7217; A61B 5/0205; A61B 5/725; A61B 5/11; A61B 5/0059; A61B 5/7278

(57) ABSTRACT

Heart rate monitors are plagued by noisy sensor data, which makes it difficult for the monitors to output a consistently accurate heart rate reading. To address the issue of noise, some monitors blindly discard sensor data which are too noisy, and stop producing heart rate readings. In some cases, if the monitors do not discard the noisy sensor data, the noisy sensor data can cause irregular heart rate readings. As a result, noisy data can lead to inaccurate heart rate readings or no heart rate readings at all. The present disclosure describes an improved technique for qualifying an input signal, i.e., determining whether a portion of the input signal is likely to result in an accurate heart rate reading, by assessing whether the frequency information of the input signal resembles a heartbeat. The resulting improved heart rate monitor is robust in tracking the heart rate in a noisy environment.

### TECHNICAL FIELD OF THE DISCLOSURE

The present invention relates to the field of digital signal processing, in particular to digital signal processing for tracking a slow moving frequency in a noisy environment.

### BACKGROUND

Modern electronics are ubiquitous in healthcare. For example, monitoring equipment systems are often provided with electronic components and algorithms to sense, measure, and monitor living beings. Monitoring equipment can measure vital signs such as respiration rate, oxygen level in the blood, heart rate, and so on. Not only monitoring equipment are used in the clinical setting, monitoring equipment are also used often in sports equipment and consumer electronics.

One important measurement performed by many of the monitoring equipment is heart rate, typically measured in beats per minute (BPM). Athletes use heart rate monitors to get immediate feedback on a workout, while health care professionals use heart rate monitors to monitor the health of a patient. Many solutions for measuring heart rate are available on the market today. For instance, electronic heart rate monitors can be found in the form of chest straps and watches. However, these electronic heart rate monitors are often not very accurate, due to a high amount of noise present in signals provided by the sensors of these monitors. The noise is often caused by the moving user and lack of secure contact between the monitor and the user. The noisy environment often lead to a lack of a BPM output, or an irregular/abnormal BPM output.

### Overview

Heart rate monitors are plagued by noisy sensor data, which makes it difficult for the monitors to output a consistently accurate heart rate reading. To address the issue of noise, some monitors blindly discard sensor data which are too noisy, and stop producing heart rate readings. In some cases, if the monitors do not discard the noisy sensor data, the noisy sensor data can cause irregular heart rate readings. As a result, noisy data can lead to inaccurate heart rate readings or no heart rate readings at all. The present disclosure describes an improved technique for qualifying an input signal, i.e., determining whether a portion of the input signal is likely to result in an accurate heart rate reading, by assessing whether the frequency information of the input signal resembles a heartbeat. The resulting improved heart rate monitor is robust in tracking the heart rate in a noisy environment.

### BRIEF DESCRIPTION OF THE DRAWING

To provide a more complete understanding of the present disclosure and features and advantages thereof, reference is made to the following description, taken in conjunction with the accompanying figures, wherein like reference numerals represent like parts, in which:

FIG. 1 shows an illustrative heart rate monitoring apparatus and a portion of a living being adjacent to the heart rate monitor, according to some embodiments of the disclosure.

FIG. 2 illustrate a system view of a heart rate monitoring apparatus, according to some embodiments of the disclosure;

FIG. 3 illustrates an exemplary flow diagram of a method for tracking a slow varying frequency present in one or more input signals provided by one or more sensors in a noisy environment, according to some embodiments of the disclosure;

FIG. 4 illustrates an exemplary flow diagram of a more detailed method for tracking a slow varying frequency present in one or more input signals provided by one or more sensors in a noisy environment, according to some embodiments of the disclosure;

FIG. 5 illustrates possible heartbeat qualifiers and signal qualifiers usable in an improved mechanism for qualifying an input signal, according to some embodiments of the disclosure;

FIGS. 6A-C illustrate an exemplary implementation of a method for tracking a slow varying frequency present in one or more input signals provided by one or more sensors in a noisy environment, according to some embodiments of the disclosure;

FIGS. 7A-B show a relatively good data set with minor noise and exemplary results by applying the improved method for tracking a slow varying frequency on such a data set, respectively, according to some embodiments of the disclosure;

FIGS. 8A-B show a data set with a large noisy portion and exemplary results by applying the improved method for tracking a slow varying frequency on such a data set, respectively, according to some embodiments of the disclosure; and

FIGS. 9A-B show a data set with a portion of data exhibiting saturation and a large amount of noise and exemplary results by applying the improved method for tracking a slow varying frequency on such a data set, respectively, according to some embodiments of the disclosure.

### DESCRIPTION OF EXAMPLE EMBODIMENTS OF THE DISCLOSURE

Understanding Issues of Noisy Environment of Heart Rate Monitors

Heart rate monitors are often provided adjacent to the skin of a living being. The monitors passively track or measure heart rate by sensing one or more aspects of the skin adjacent to the heart rate monitor. Due to the passive nature of such measurements, the sensor data can be affected by many sources of noise which severely affect the ability of the heart rate monitor in determining an accurate heartbeat. These sources can include external interference to the sensor, internal noise of the sensor and/or heart rate monitor, motion causing disruptions in the sensor's capability in measuring the aspects of the skin, etc. Furthermore, heart rate monitors are affected by variability in the skin of different living beings and the variability of the skin and environment during the use of the heart rate monitor. All these different sources and issues have adverse impact on the heart rate monitor's ability to extract an accurate heart rate.

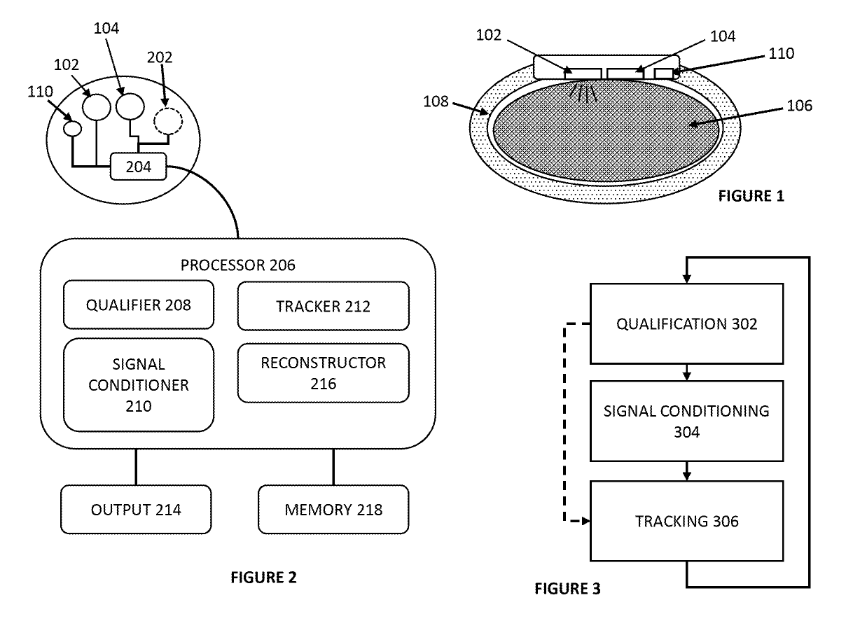

The present disclosure describes some specific challenges faced by heart rate monitors using a light source and an optical sensor to measure a heart rate of a living being. FIG. 1 shows an illustrative heart rate monitoring apparatus and a portion of a living being adjacent to the heart rate monitor, according to some embodiments of the disclosure. In particular, the FIGURE shows a cross section to illustrate the monitoring apparatus's spatial relationship with the portion of the living being. In this exemplary heart rate monitoring setup, a method of photophethysmography (PPG) is used, where the heart rate is measured passively or indirectly based on changes in light absorption in the skin as blood is pushed through the arteries. If the signal provided by the optical sensor 104 does not have a lot of noise, pulses in the signal as a result from the changes in blood volume as blood is pumped through the arteries can be observed. The pulses in the signal can then be used in extracting a heart rate.

Heart rate monitoring apparatus described herein are not limited to the particular example shown in FIG. 1. Although the disclosure does not describe other types of heart rate monitors in detail, one skilled in the art would appreciate that these challenges are also applicable in other types of heart rate monitors or other types of devices providing heart rate monitoring functions, or even devices utilizing other types of sensing mechanism. Furthermore, the continued process of measuring, following, extracting, determining, or sensing the heart rate (or some other slow varying frequency) over time is referred to as “tracking a slow varying frequency”, within the context of the disclosure.

Specifically, FIG. 1 illustrates an exemplary heart rate monitoring apparatus having a light source 102 and an optical sensor 104. The light source can emit light within a range of wavelengths suitable for the application. In some embodiments, the light source 102 and the optical sensor 102 can be provided separately, or a light source 102 can be biased to perform as an optical sensor 104. For instance, a red LED can be used as a red light source and a red optical detector. In some embodiments, both the light source 103 and optical sensor 104 can be provided nearby each other in a housing or member of the heart rate monitoring apparatus or in any suitable configuration where the optical sensor 104 can measure absorption of light (as generated by the light source 103) by the part 106 of the living being. The light source shines a light onto a part 106 of a living being 106 and the optical sensor 104 measures light near the optical sensor 104, which can include light being reflected from the part 106 and ambient light. Various parts of the living being can be used as part 106, e.g., a finger, an arm, a forehead, an ear, chest, a leg, a toe, etc., as long as changes in the volume of blood can be measured relatively easily. The part 106 can in some cases be internal to the body of the living being.

Generally speaking, if the heart rate monitoring apparatus can be affixed to the part 106 of the living being securely and maintain relatively stable contact with the part 106 during use, the input signal provided by the optical sensor would exhibit very little noise and the heart rate can be easily extracted. However, in many scenarios, the heart rate monitoring apparatus is not securely affixed to the part 106 (even with the use of part 108 involving a band, a strap, adhesive, or other suitable attachments), or having the apparatus securely adhered or attached to the part 106 is not desirable or comfortable for the living being. In these scenarios, the signal provided by the optical sensor 104 is usually affected by noise from ambient light, artifacts caused by motion of the heart rate monitoring apparatus, or by some other noise source. As a result, correctly detecting the heart rate in these non-ideal scenarios, i.e., in a noisy environment, can be challenging. Attempting to detect the heart rate based on a noisy signal can result in irregular or erroneous heart rate readings.

To address this issue, some heart rate monitoring apparatuses include a mechanism which discards certain portions of data if the data is deemed unusable for tracking heart rate. The mechanism can include an accelerometer 110 to measure the motion of the apparatus to assess whether the input signal is likely to be too degraded by motion artifacts to be relied upon for heart rate determination. In those cases, the accelerometer reading can cause the apparatus to discard data when the accelerometer 110 senses too much motion or use accelerometer data to estimate the heart rate. This can be problematic for heart rate monitoring apparatuses which experiences a lot of acceleration (e.g., in a sports setting), and the user would simply not have a heart rate output, or an accurate heart rate output would not be available during a substantial amount of time during use. An even more subtle problem for such apparatuses is that the blindly discarded portions of signal data can still be good enough for heart rate detection, but the blindly discarded portions are no longer used for tracking.

Some heart rate monitoring apparatuses discards portions of the signal which is deemed too noisy by assessing signal quality (e.g., how clear spectral peaks are in the frequency domain). This could be helpful in removing noisy portions of the signal, but the data which had not been discarded is not always reliable for heartbeat tracking. While such apparatuses can discard a portion of the signal that is too noisy, certain portions of the input signal exhibiting clear spectral peaks used for tracking the heart beat can still result in erroneous heart beat readings because the clear peaks could have been a result of motion artifacts or other sources of artifacts affecting heart rate detection. For instance, a portion of the input signal degraded by motion artifacts but having clear spectral peaks could cause a heart rate tracking mechanism to track onto a frequency corresponding to the motion artifact and not to the true heart rate.

Improved System and Method for Tracking a Slow Varying Frequency

The aforementioned problems of heart rate monitoring apparatuses stem from having a coarse mechanism for discarding input data. In other words, the failures of the aforementioned apparatuses are caused by the coarse mechanism not being able to precisely distinguish between a good signal, a bad signal, and a somewhat bad signal in a nuanced way. The present disclosure describes an improved qualification mechanism which alleviates some of the issues mentioned above. The improved qualification mechanism is more nuanced and can enable the input signal to be conditioned in such a way to allow the tracker to track the heart rate better even in the presence of noise. By improving on the “qualification” mechanism, the heart rate monitoring apparatus can achieve more robust performance in a noisy environment. An improved “qualification” mechanism can increase the amount of the usable data of input signal and thereby increase the accuracy and consistency of heart rate output. Furthermore, the improved “qualification” mechanism can improve the accuracy of the tracking mechanism for tracking the heart beat by way of providing a better and more usable input signal.

The improved qualification mechanism leverages characteristics of a heart rate to better determine which portions of the input signal should be discarded or attenuated. Specifically, the improved qualification mechanism aims to keep portions of the input signal which resembles a heartbeat based on the insights that:

A heart rate is typically does not go away;

A heart rate is slow varying, i.e., the heart rate changes relatively slowly over time; and

A heart rate is typically confined to 0.5 Hz-3.5 Hz.

In particular, the improved qualification mechanism leverages the last insight related to the slow varying aspect of a heartbeat to attenuate/discard data samples which do not exhibit a slow varying frequency. The resulting qualification mechanism is able to better qualify the input signal and improve the accuracy of heart rate tracking. The following passages describe in further detail how the improved qualification mechanism can be implemented and realized.

An Exemplary Improved Heart Rate Monitoring Apparatus and Method

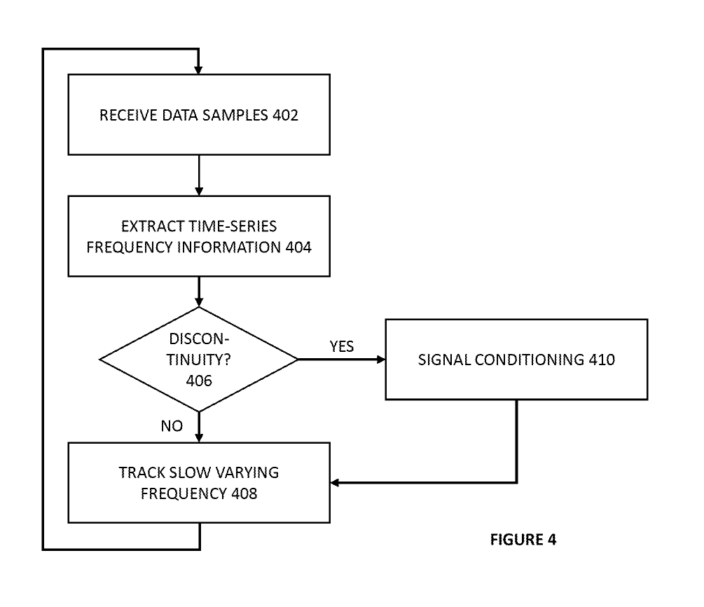

FIG. 2 illustrate a system view of a heart rate monitoring apparatus, according to some embodiments of the disclosure. The system provides an arrangement of parts for implementing or enabling a method for tracking a slow varying frequency present in one or more input signals provided by one or more sensors in a noisy environment. Similar to FIG. 1, the apparatus includes a light source 102, an optical sensor 104. The light source can be a light emitting diode (LED), or any suitable component for emitting light. The light emitted by the light source 102 for measuring heart rate (e.g., blood volume) can be any suitable wavelength depending on the application. The apparatus can include a plurality of light sources emitting a range of wavelengths of light. The optical sensor 104 can be the same device as the light source 102, or the optical sensor 104 is provided near the light source 102 to measure light near the optical sensor 104, e.g., to measure absorption of light emitted by the light source 102 in the skin to implement PPG. Optionally, the apparatus can include accelerometer 110 to measure acceleration of the overall apparatus. Furthermore, the apparatus can include other sensors 202 or other types of sensors, which can provide information to assist in qualification and/or heart rate tracking. An integrated circuit 204 can be provided to drive the light source 102 and provide an analog front end 204 to receive signals provided by optical sensor 104, accelerometer 110, and other sensors 202. In some embodiments, the analog front end 204 can convert (if desired) analog input signals to data samples of the analog input signal. The analog front end can be communicate with a processor 206 to provide the data samples, which the processor 206 would process to track a slow varying frequency, e.g., the heartbeat.

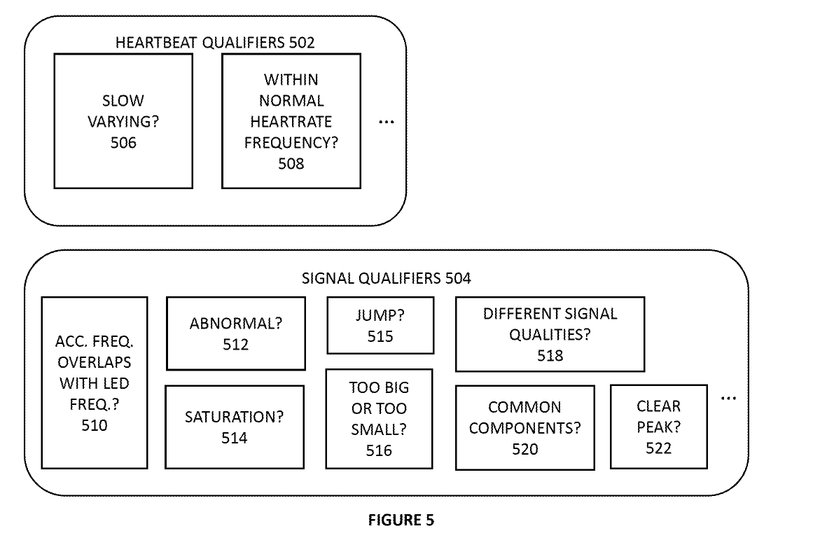

The processor 206 can include several special application specific parts or modules for processing the data samples of the input signal to track the slow varying frequency. The processor 206 can include electronic circuits specially arranged to processing the data samples of the input signal to track the slow varying frequency. The processor 206 include programmable logic gates specially arranged to process the data samples of the input signal to track the slow varying frequency. The processor 206 can be a digital signal processor provided with application specific components to track the slow varying frequency, and/or the processor can execute special instructions (stored on non-transitory computer readable-medium) for carrying out the method of tracking the slow varying frequency. FIG. 3 illustrates an exemplary flow diagram of such a method, e.g., that the apparatus shown in FIG. 2 can implement using the processor 206, for tracking a slow varying frequency present in one or more input signals provided by one or more sensors in a noisy environment, according to some embodiments of the disclosure. At a high level, the method includes a qualification component 302, a signal conditioning component 304 (dependent on the qualification component 302), and a tracking component 306 (dependent on the qualification component 302 and/or the signal conditioning component 304). The method can continue back at the qualification component 302 to process other data samples in the stream of data samples of the input signal.

Referring to both FIG. 2 and FIG. 3, the parts of processor 206 can include one or more of the following: a qualifier 208, a signal conditioner 210, a tracker 212, and a reconstructor 216, e.g., to implement the method shown in FIG. 3.

The qualifier 208 implements functions related to the improved qualification mechanism (corresponding to qualification component 302 of the method shown in FIG. 3), e.g., including decision(s) which determine whether a particular portion of the data samples of the input signal is to be provided to the tracker 212, or whether a particular portion of the data samples of the input signal should be attenuated or filtered before providing the data samples to the tracker 212.

The signal conditioner 210 implement functions related to processing data samples of the input signal based on the decision(s) in the qualifier 208 to prepare the data samples for further processing by the tracker 212 (corresponding to signal conditioning component 304 of the method shown in FIG. 3). For instance, the signal conditioner 210 can filter data samples of the input signal a certain way (or apply a filter on the data samples), apply a mask to the data samples, attenuate certain data samples, modify the values of certain data samples, and/or select certain data samples from a particular sensor for further processing. The signal conditioning process can depend on the output(s) of the qualifier 208.

The tracker 212 implements functions related to tracking the slow varying frequency, e.g., the heartbeat, based on the output from the signal conditioner 210 (corresponding to tracking component 306 of the method shown in FIG. 3). In other words, the tracker continuously monitor the incoming data samples (raw data or as provided by the signal conditioner 210) and attempts to continuously determine the frequency of the slow varying frequency present in the one or more signals from the sensors. The output of the tracker 212, e.g., determined heart rate in beats per minute, can be provided to a user via output 214 (e.g., a speaker, a display, a haptic output device, etc.).

The reconstructor 216 can implement functions related to (re)constructing or synthesizing a time domain representation of the slow varying frequency, e.g., a heartbeat. Based on frequency information of the input signal, the reconstructor 216 can artificially generate a cleaner version of the input signal having the slow varying frequency (referred herein as the “reconstructed signal”). The reconstructed signal can be useful in many applications. For instance, the reconstructed signal can be provided to output 214 for display. The reconstructed signal can also be saved for later processing and/or viewing. Generally speaking, the reconstructed signal can be useful for users to visually and analytically assess the health of the living being with the irrelevant noise content removed. For instance, the reconstructed signal can assist healthcare professionals in assessing whether the living being has any underlying conditions relating to heart and arterial health. This reconstructed signal can be generated by first using the qualifier 208, the signal conditioner 210, and the tracker to track the slow varying frequency. During this process, frequency information of the improved data samples can be obtained. A peak or portion of the frequency information corresponding to the frequency of the heartbeat (the tracked slow varying frequency) can be isolated, where frequency information outside of the peak or portion is removed, including higher harmonics of the signal. The reconstructor 216 can then apply an inverse frequency transform (e.g., inverse Fast Fourier Transform) on the isolated peak to obtain the reconstructed signal.

The qualifier 208, the signal conditioner 210, the tracker 212, and the reconstructor 216 can include means for performing their corresponding functions. Data and/or instructions for performing the functions can be stored and maintained in memory 218 (which can be a non-transitory computer-readable medium). In some embodiments, the qualifier 208 (corresponding to qualification component 302 of the method shown in FIG. 3) can affect the processing performed in tracker 212 (corresponding to tracking component 306 of the method shown in FIG. 3). This feature is denoted by the arrow having the dotted line. The apparatus shown in FIG. 2 is merely an example of a heart rate apparatus, it is envisioned that other suitable arrangements can be provided to implement the improved method for tracking a slow varying frequency present in one or more input signals provided by one or more sensors in a noisy environment.

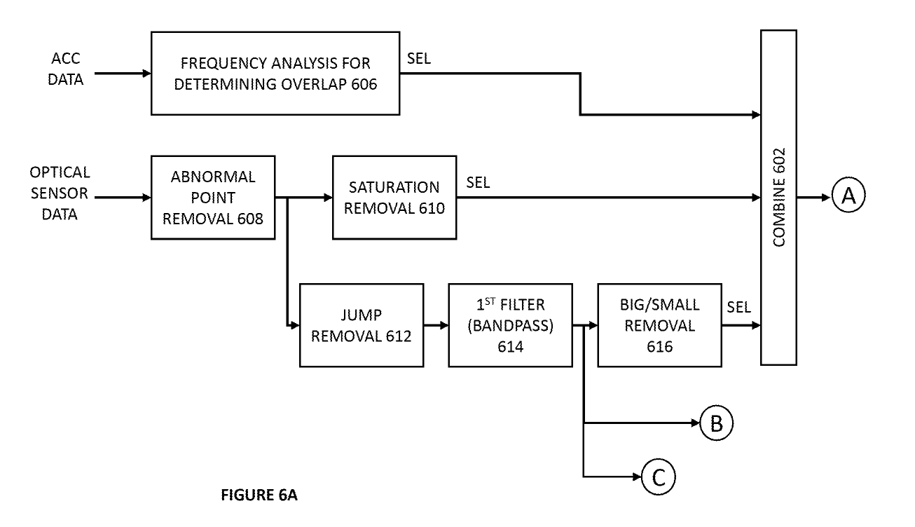

FIG. 4 illustrates an exemplary flow diagram of a more detailed method for tracking a slow varying frequency present in one or more input signals provided by one or more sensors in a noisy environment, according to some embodiments of the disclosure. One of the important aspects of the improved method for tracking a slow varying frequency present in one or more input signals provided by one or more sensors in a noisy environment is the ability to qualify when the input signal is exhibiting a slow moving frequency (or when the input is not exhibiting a slow moving frequency). This aspect advantageously prevents the tracking mechanism to lock onto other frequencies which are not associated with a heartbeat, i.e., other frequencies (often associated from other sources of noise or artifacts) which are not slow varying, or do not flow smoothly from a previously tracked slow varying frequency.

The detailed method includes receiving data samples of a first input signal (box 402). The first input signal can be generated by an optical sensor, and in some cases, the first input signal is processed by an analog front end to produce (digital) data samples of the first input signal. The data samples are received by the processor for qualification. In this detailed method, the qualification includes determining whether a slow varying frequency is present. Specifically, the detailed method further includes extracting time-series frequency information of the first input signal based on the data samples of the first input signal (box 404). For instance, a frequency transform is applied to a portion of the data samples to obtain frequency information (relating a range of frequencies and amplitudes for the range of frequencies). Prominent components of the frequency information (e.g., harmonics, peaks or frequencies having a large amplitude) can indicate frequencies of periodic signals in the input signal, such as a heart rate. Furthermore, the detailed method includes determining whether the frequency information exhibits discontinuities in the time-series (diamond 406).

Leveraging the insight that a heart rate is slow varying, the frequency information, e.g., the prominent components observed in the time-series frequency information, is examined to determine whether there is a slow varying frequency (over time). Discontinuities or disruptions in the time-series frequency information can indicate that the prominent components in the frequency information is more likely evidence of other sources which are not associated with a heartbeat.

This insight allows the tracking of the slow varying frequency to be performed in a more intelligent way, where the method can process the data samples to track the slow varying frequency based on whether the frequency information exhibits discontinuities. If discontinuity is observed in the time-series frequency information (the prominent components jumps from one frequency to another frequency over a short period of time, i.e., the frequency is not slow varying), the method can perform signal conditioning of the data samples of the input signal to, e.g., filter, remove, or mask, portions of the data samples which are likely not associated with a heartbeat (box 410) before tracking the slow varying frequency (box 408). If no (sharp) discontinuity is observed in the time-series frequency information (the prominent components only varies from one frequency to another frequency over a long period of time, or the frequency is slow varying), the method proceeds to perform tracking of the slow varying frequency (box 408).

One important advantage of this improved qualification mechanism is its ability to prevent other signals or noise sources of a different frequency to be falsely detected as the slow varying frequency, i.e., the heartbeat. The qualification allows proper signal conditioning to prepare data samples that can lead to better tracking. This advantage can achieved by filtering, removing, attenuating, modifying, or removing certain undesirable portions of the data samples prior to the data samples being processed to track the slow varying frequency.

While many examples described herein are described in relation to a slow varying frequency representative of a heartbeat, it is envisioned that the method can be applicable in other scenarios for tracking other types of slow varying frequencies (e.g., phenomena or events which has a frequency that does not change or jump abruptly). Furthermore, while the examples herein are described with one or more input signals provided by one or more optical sensors, it is envisioned that the method can be used to track a slow varying frequency present in signals generated by other types of sensors, including but not limited to: optical sensor, audio sensor, capacitive sensor, magnetic sensor, chemical sensor, humidity sensor, moisture sensor, pressure sensor, and biosensor.

Heartbeat Qualifiers and Signal Qualifiers

Besides leveraging the slow varying frequency of a heart rate, there are several other ways to improve qualification (to enable better signal conditioning of data sample prior to tracking). Broadly speaking, qualifiers can intelligently identify portions of the data samples of the input signal which would be more likely to result in accurate and consistent tracking of a heartbeat. When used alone or in combination, the decisions made using the qualifiers can control signal conditioning of the data samples prior to the data samples being processed by the tracker (i.e., prior to trying to extract or determine the frequency corresponding to the slow varying frequency present in the one or more input signals). As a result, the qualifiers can improve the quality of the data samples being provided to the tracker, and the tracking would become more robust in the presence of noise.

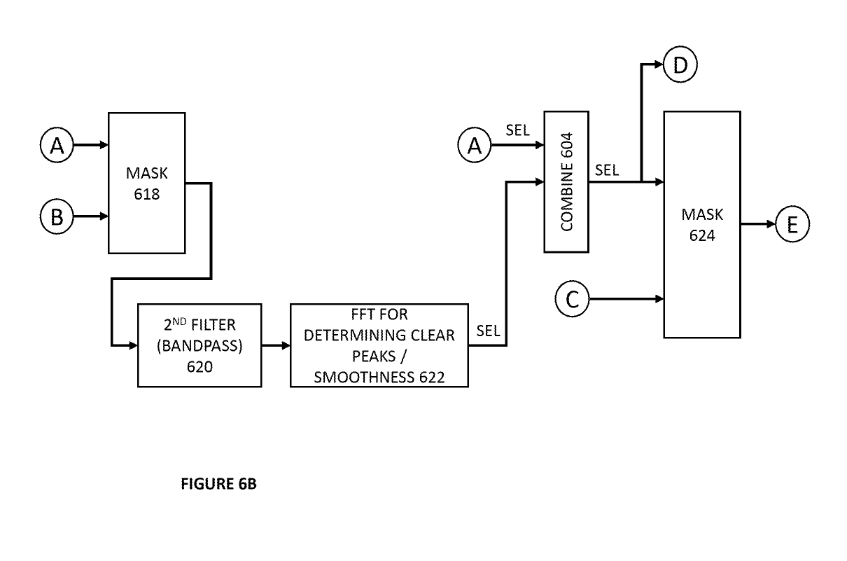

FIG. 5 illustrates possible heartbeat qualifiers and signal qualifier usable in an improved mechanism for qualifying an input signal, according to some embodiments of the disclosure. Heartbeat qualifiers leverage characteristics of a heartbeat to assess how likely the one or more input signals has a heartbeat, and signal qualifiers infer from one or more input signals whether the one or more input signals is too corrupted by noise and/or artifacts to enable a heart rate to be accurately and consistently tracked. The categorization of heartbeat qualifier versus signal qualifiers is not definitive, as some heartbeat qualifiers can be related to signal qualifiers.

A plurality of qualifiers can be used to process data samples of the input signal to assess whether the data samples are suitable for tracking or to control how the signal conditioning should process the data samples to prepare them for tracking. One or more qualifiers can also control the tracking process, if desired. The outputs from the qualifiers can be combined using an AND operator, OR operator, a voting mechanism, a weighted voting mechanism, a decision tree, an artificial intelligence algorithm for classifying whether the data samples are good or bad, any suitable combination mechanisms for combining outputs from the qualifiers, etc. The outputs can cause certain portions of the data samples to be masked or attenuated, or the data samples to be filtered a certain way to prepare the data samples for tracking. In some cases, the outputs of the qualifiers are not combined together prior to being provided to the signal conditioning process, but are fed as inputs or parameters to the signal conditioning process to control such process.

Heartbeat Qualifier: Slow Varying Frequency Over Time

In one example, the improved qualification mechanism includes a qualifier (“SLOW VARYING?” 506) for determining whether time-series frequency information exhibits discontinuities, or conversely, whether the time-series frequency information exhibits a slow varying frequency over time (e.g., using a method described in relation to FIG. 4). This qualifier relates to the method described in FIG. 4. Detecting discontinuities in the frequency information (i.e., detecting an abrupt change or large change in a prominent frequency which had been slow varying) is a good indicator that the data samples is likely to include signal components which are not related to a heartbeat. In response to the qualification, the overall tracking method can include applying a filter or mask to or removing a portion of the data samples which is associated with a discontinuity in the frequency information prior to processing the data samples to track the slow varying frequency (to better condition the signal for tracking).

In some implementation, the “SLOW VARYING?” qualifier 506 can extract time-series frequency information of the first input signal by determining frequency information of a first window of data samples and frequency information of a second window of data samples. The windows can be (highly) overlapping or substantially adjacent to each other, depending on the implementation. Furthermore, the qualification mechanism can determine whether the frequency information exhibits discontinuities in the time-series by determining whether a difference between the frequency information of the first window of data samples and frequency information of the second window of data samples is greater than a threshold. For instance, the qualifier can compute the difference between one prominent component of the frequency information of the first window and one prominent component of the frequency information of the second window to assess whether the difference is greater than the threshold.

The threshold can be provided in different ways. The threshold can be predetermined empirically based on past test data. In some cases the threshold is adaptive, e.g., the threshold can be based on an average of past frequencies associated with prominent components in the time-series frequency information and/or changes in the past frequencies associated with prominent components in the time-series frequency information. The adaptive threshold can advantageously assess whether a change in frequency is too big of a discontinuity based on past variations in the past frequencies associated with prominent components of the time-series frequency information. The threshold can be parameterizable based on one or more factors or user input.

Heartbeat Qualifier: A Reasonable Frequency Band of Interest

In one example, the improved qualification mechanism includes a qualifier (“WITHIN NORMAL HEARTRATE FREQUENCY?” 508) for determining whether the frequency information includes prominent components outside of an expected range of frequencies representative of the slow varying frequency the method is tracking (i.e., a heartbeat). Typically, a heart rate is between 0.5 Hertz to 3.5 Hertz (in some cases it can be as high as 4 or 5 Hertz). If the frequency information of the input signal has prominent components outside of the reasonable frequency band of interest, it is likely the data samples do not have a trackable heartbeat.

This qualifier can be incorporated with a signal conditioning process by processing the data samples with a filter to substantially attenuate signal content outside of a reasonable frequency band of interest corresponding to the slow varying frequency of the input signal (or apply a masking process to achieve a similar effect) before extracting time-series frequency information of the input signal. The filter can be a low-pass filter (e.g., passing signals in a bandwidth from 0-3.5 Hertz, 0-4 Hertz, 0-4.5 Hertz or similar variant thereof) or a band-pass filter (e.g., passing signals in the bandwidth from 0.5-3.5 Hertz, 0.5-4 Hertz, 0-4.5 Hertz, or similar variant thereof). The type of filter used to attenuate signals outside of the reasonable frequency band of interest can vary depending on the application. Furthermore, the reasonable frequency band of interest can vary depending on the application. In one example, the reasonable frequency band of interest includes a frequency band of 0.5 Hertz to 3.5 Hertz (or includes frequencies between 0.5 Hertz to 3.5 Hertz), which is suitable for keeping frequency content that is more likely to be associate with a heartbeat.

Signal Qualifier: Accelerometer Assisted Qualification

If an accelerometer is provided as part of the heart rate monitoring apparatus, it is possible to infer information about the data samples of the input signal provided by the optical sensor based on frequency information of the accelerometer data. For example, it is possible to determine whether the data samples are still usable for tracking even when there is a lot of motion. While some monitoring apparatuses assume that no input signal from the optical sensor is usable if there is a lot of motion, the reality is simply not true because the optical sensor can still report some usable signal in the presence of motion, especially if the contribution from motion in the input signal of the optical sensor occurs at a different frequency from the reasonable frequency expected from a heart rate (0.5-3.5 Hertz). For this reason, an improved qualification mechanism can include a qualifier (“ACC. FREQ. OVERLAPS WITH LED FREQ.?” qualifier 510) for determining whether the data samples of the input signal provided by the optical sensor is reporting too much motion to be usable by the tracker, or if the input signal is still usable by the tracker because it is not severely affected by motion.

Rather than simply discarding a portion of the data samples when the accelerometer has detected a lot of motion, the qualifier determines if there are common components/harmonics between the frequency information of the data samples of the input signal provided by the optical sensor and the frequency information of the data samples of the input signal provided by the accelerometer. If prominent components of frequency information associated with motion data is also appearing in the frequency information associated with optical sensor data, a tracker could erroneously track onto the prominent components which is probably a result of motion artifacts instead of the real heartbeat. The method for tracking can thus include receiving data samples of a signal indicative of motion of the one or more sensors and extracting time-series motion frequency information based on the data samples of the signal indicative of motion. When incorporated with signal conditioning, the method further includes, applying a filter or mask to or removing a portion of the data samples associated with the same particular time prior to processing the data samples to track the slow varying frequency, if the motion frequency information corresponding to a particular time has one or more components common with frequency information corresponding to the same particular time.

Signal Qualifier: Abnormalities

Sometimes noise, errors, or faults in the sensor or other electronics supporting the sensors can generate abnormal values. An improved qualification mechanism can include a qualifier (“ABNORMALITIES”? qualifier 512) for determining which data samples are too abnormal. Data samples can be deemed to be abnormal if they are greater than a predetermined threshold. The predetermined threshold can be established based on a known range of values that the sensors is able to generate. For instance, the threshold can set an absolute boundary of for determining when a data value is too high or too low, or when the data value is beyond a predetermined expected range of values. For instance, if a sensor is rated to only generate values between −10 to +10, a value of 100 is abnormal (e.g., based on a threshold of +10, or +10 plus or minus a reasonable range of acceptable error).

Specifically, the qualifier may examine the values of the data samples, and based on a criteria, such as a threshold, assess whether a particular data sample is abnormal (e.g., having a value that is likely caused by noise or some other artifact). For instance, certain values of data samples are unusual and are likely to indicate an error in the data values. If a data sample has a value which exceeds the threshold or below the threshold, it is determined that the data sample is associated with an abnormal condition. When incorporated with signal conditioning, the method for tracking a slow varying frequency further includes applying a filter or mask to or removing a portion of the data samples indicative of an abnormal condition of the one or more sensors prior to processing the data samples to track the slow varying frequency. For instance, the abnormal data samples can be removed, or replaced with normal values, or previous/subsequent values.

Signal Qualifier: Saturation

An improved qualification mechanism can include a qualifier (“SATURATION”? qualifier 514) for determining when data samples appear to be in saturation. Saturation can occur due to the underlying physical characteristics of sensors and electronic circuits. When saturation occurs, the input signal would appear to “flat line” and would appear to be distorted. For instance, data samples may include the following sequence of values: [1, 2, 2, 3, 4, 5, 4, 5, 6, 7, 9, 9, 9, 9, 9, 9, 9, 6, 7, 6, 5, 4, 4, 4, 3, 2, 4]. The sequence of 9's appear to indicate a saturation condition. When the input signal is saturated, the data samples are likely to not provide any good information for tracking.

To qualify data samples as being saturated, the qualifier may examine the values of the data samples. For instance, the qualifier may examine whether the data values has not changed for a period of time (or a number of samples). If the data values has not changed for a period of time, it is likely the input signal is saturated. Based on a saturation criteria, the qualifier can assess whether a particular data sample is associated with a saturation condition. When incorporated with signal conditioning, the method for tracking a slow varying frequency further includes applying a filter or mask to or removing a portion of the data samples indicative of a saturation condition of the one or more sensors prior to processing the data samples to track the slow varying frequency.

Signal Qualifier: Jump (or Offset)

An improved qualification mechanism can include a qualifier (“JUMP”? qualifier 515) for determining when data samples appear to change too abruptly and remains shifted by an offset for a period of time. For instance, data samples may include the following sequence of values: [1, 1, 1, 2, 3, 2, 1, 27, 29, 28, 27, 28, 27, 28, 3, 2, 2, 1, 2, 3, 4]. The sequence of values [27, 29, 28, 27, 28, 27, 28] appear to have an offset different from sequences [1, 1, 1, 2, 3, 2, 1] and [3, 2, 2, 1, 2, 3, 4]. An ideal signal without a lot of noise or artifacts from a sensor sensing a heart rate should be relatively slow varying and smooth, and not experience an offset in the values abruptly. For this reason, when values of data samples jumps to another offset for a period of time, the data samples are likely to not provide any good information for tracking, in absence of any signal conditioning.

To qualify data samples as having a shift or jump in the offset, the qualifier may examine the values of the data samples. For instance, the qualifier may examine whether the difference(s) between data samples are too high (or exceeds a certain threshold or exceeds an allowable amount of change) for a number of samples. In some instances, the qualifier may examiner certain adjacent or highly non-overlapping windows of data samples and average values of those windows of data samples to assess whether the values of the data samples have shifted by an offset. If offset observed is greater than a predetermined threshold, it is likely the input signal is corrupted.

When incorporated with signal conditioning, the method can include applying a filter or mask to or removing a portion of the data samples which are associated with an offset exceeding a predetermined threshold prior to processing the data samples to track the slow varying frequency. For instance, the values of the data samples can be shifted to remove the jump in the offset. In another instance, a low pass filter or smoothing filter can be applied to attenuate the magnitude of the offset change in the data values. Other ways of removing the jump are envisioned. Following the example above, the sequence of values [27, 29, 28, 27, 28, 27, 28] with the offset (e.g., caused by an artifact) artificially removed, can become [3, 5, 4, 3, 4, 3, 4] (subtracting an offset of 24), or [4, 6, 5, 4, 5, 4, 5] (subtracting an offset of 23).

Signal Qualifier: Too Big or Too Small

An improved qualification mechanism can include a qualifier (“TOO BIG OR TOO SMALL”? qualifier 516) for determining when data samples appear to be too large or too small. An ideal signal from a sensor sensing a heart rate should fall within a range of reasonable values based on values of past or subsequent data samples. For this reason, when values of data samples has values outside of the range or is too far from an average value of the data samples, the data samples are likely to not provide any good information for tracking, as the data samples are likely to be corrupted by noise or some other artifact. The range of reasonable values can vary depending on values of previous and/or subsequent data samples, and the range can be determined empirically based on test data. The range can also be determined based on an average value of a window of previous and/or subsequent data samples.

To qualify data samples as being too big or too small, the qualifier may examine the values of the data samples. For instance, the qualifier may examine whether a value of a data sample falls outside of the range of expected values computed based on previous and/or subsequent data samples. In some instances, the qualifier may determine whether the difference between the value of the data sample and an expected average value is above a threshold. If the value of a data sample falls outside of the range (an expected range computed from previous and/or subsequent data samples), it is likely the input signal is corrupted. When incorporated with signal conditioning, the method for tracking a slow varying frequency can include applying a filter or mask to or removing a portion of the data samples which is outside of an expected range of values prior to processing the data samples to track the slow varying frequency. In some instances, a mask or filter can replace the data sample outside of the expected range of values with the expected average value.

Using More than One Optical Sensor

The wavelengths used for measuring input signals for PPG can span wavelengths from blue to infrared. In classic applications, LEDs of two colors—often 660 nm and 940 nm—are used for measuring blood oxygen saturation. These devices are in large volume production and are readily available. In yet another application, a simple single-color LED—say at 940 nm, may be used to measure heart rate by measuring the periodic variation in a return signal. In some cases, a green LED is used to pick up variation in absorption caused by blood flow on the wrist.

Different wavelengths of light reflects differently from skin (due to the pigmentation and wrinkles, and other features of the skin) and different optical sensors tend to behave differently in the presence of motion when sensing light reflected from skin. Based on this insight, it is possible to infer information about presence of motion and/or the quality of an input signal. It is also possible to improve the data samples to be processed by the tracker based on the insight. Multiple light sources having different wavelengths can be used (e.g., a red LED and a green LED). For instance, by sensing these light sources and examining differences between the input signals of optical sensors for detecting light having respective wavelengths, or different portions of a spectrum of an input signal from a wideband optical sensor, it is possible to infer whether certain data samples of the input signal is likely to have been affected by motion or some other artifact.

Broadly speaking, an internally consistent model can be provided if different characteristics and behavior of different types of optical sensors under various conditions (or in general) are known. Based on the internally consistent model, information about the signal or the environment of the sensors can be inferred. The inference can assist qualifiers in assessing whether certain portions of the data samples should be removed. The inference can also assist signal conditioning to specify how the data samples should be processed to improve tracking. This can include filtering the signal a certain way. The inference can also, in some cases, signal to the tracker to perform tracking differently.

In some instances, the use of multiple optical sensors can improve tracking by removing or subtracting common global characteristics between optical sensors to better track the slow varying frequency. In some cases, the internally consistent model may prescribe that the tracked slow varying frequency (e.g., the tracked heart rate) should be substantially the same for a plurality of sensors (e.g., the red LED should measure the same heart rate as the green LED). The following passage describes some exemplary internally consistent models that can be used to improve tracking of the slow varying frequency.

Signal Qualifier: Multiple Optical Sensors Providing Input Signals with Different Signal Qualities

An improved qualification mechanism can include a qualifier (“DIFFERENT SIGNAL QUALITIES” qualifier 518) to assess differences in signal quality between input signals from different optical sensors. Some input signals provided by certain optical sensors tend to degrade more quickly in the presence of motion. For instance, when there is motion and a red LED and green LED are both used, the input signal from the red LED would degrade faster than the input signal from the green LED. If this condition was detected, the qualifier can infer that motion is present, and signal conditioning can take appropriate action or the tracking can adapt based on the inference.

Based on this inference, the method for tracking a slow varying frequency can include receiving data samples of a second input signal, wherein the first input signal is provided by a first optical sensor has a first signal quality that is different, in presence of motion or source of noise, from a second signal quality of the second input signal provided by a second optical sensor. The qualifier can determine whether signal quality of the first input signal and signal quality of the second input signal are substantially different for a particular time. For instance, the qualifier can measure signal-to-noise ratios of the two input signals or some other indicator of noise level. A tracker can processing the data samples to track the slow varying frequency based on whether signal quality of the first input signal and signal quality of the second input signal are substantially different for a particular time. For instance, if a tracker has a different operating modes, the tracker can switch to an operating mode that is more suitable for processing the data samples of the input signal in the presence of motion, or an operating mode that is more robust against motion artifacts. In another instance, the tracker can use the data samples from the better input signal (having the higher signal quality or higher signal to noise ratio) for tracking. In another instance, the detection of a difference in signal quality between the two input signals can cause certain data samples associated with the period when the signal qualities are different to be filtered, masked or attenuated a certain way.

Signal Qualifier: Multiple Optical Sensors Having Common Components

Generally speaking, different optical sensors associated with different wavelengths has different behavior in response to small movements (e.g., due to wrinkles or hair on the skin, inherently different absorptivity of the particular wavelength in the skin). But the behavior of the different optical sensors should have consistent behavior in response to big global movements (e.g., larger movements experienced by both optical sensors at the same time). A good tracker can detect the slow varying frequency in the presence of small movements, but trackers may not perform very well in the presence of big global movements, since the signals from the optical sensors are unlikely to be very meaningful. If there are common prominent components in the frequency information of one input signal provided by, e.g., a red LED, and in the frequency information of another input signal provided by, e.g., a green LED, the common prominent components present in the frequency information are more likely to have resulted from big global movements and not likely to be associated with a heart rate.

Leveraging this insight, an improved qualification mechanism can include a qualifier (“COMMON COMPONENT” qualifier 520) for determining whether there are common components between the two different input signals. If this condition is detected, the qualifier can infer that motion is present, and signal conditioning can take appropriate action or the tracking can adapt based on the inference. The method for tracking a slow varying frequency can include receiving data samples of a second input signal, wherein the first input signal is provided by a first optical sensor for sensing a first bandwidth and the second input signal is provided by a second optical sensor for sensing a second bandwidth different from the first bandwidth. A qualifier can extracting time-series frequency information based on the data samples of the second input signal, and assess whether there are common components between the frequency information from the two different input signals. For instance, a red LED and a green LED can be used for generating the two different input signals sensing light of different wavelengths.

When combined with signal conditioning, the method can include applying a filter or mask to or removing a portion of the data samples associated with the same particular time prior to processing the data samples to track the slow varying frequency, if the frequency information of the first input signal corresponding to a particular time has one or more components common with frequency information of the second input signal corresponding to same particular time. In some cases, a tracker can process the data samples to track the slow varying frequency based on whether the frequency information of the first input signal corresponding to a particular time has one or more components common with frequency information of the second input signal corresponding to the same particular time. For instance, if a tracker has a different operating modes, the tracker can switch to an operating mode that is more suitable for processing the data samples of the input signal in the presence of motion. In another instance, a signal can be provided to the tracker to not update the slow varying frequency to the one or more common components as they are likely not associated with the slow varying frequency the tracker is aiming to track.

Signal Qualifier: Clear Peaks

An improved qualification mechanism can include a qualifier (“CLEAR PEAKS” qualifier 522) for determining whether the signal has sufficiently low enough noise for tracking. Tracking often depend on finding and following prominent components in the frequency domain, and if the frequency information does not have clear peaks, the ability to perform tracking is degraded. If this condition is detected, the qualifier can infer that the input signal is too noisy, and signal conditioning can take appropriate action or the tracking can adapt based on the inference. The method for tracking a slow varying frequency can include determining whether peaks are sharp or clear enough in the frequency information of the first input signal. For instance, the sharpness of peaks in the frequency information can be compared against a sharpness threshold. In some cases, the number of peaks can be counted to assess whether there are too many small peaks, which is also an indicator that there is a lot of noise in the input signal.

When combined with signal conditioning, the method can include applying a filter or mask to or removing a portion of the data samples associated with the same particular time prior to processing the data samples to track the slow varying frequency if sharp peaks are not detected. In some implementations, a sliding window method can be used to obtain the time-series frequency information. Each sliding window having a particular data sample can provide a vote for determining whether that data sample is associated with frequency information having clear peaks. The number of votes accumulated for the particular data sample can be used as part of the qualifier in determining whether the data sample should be masked, or attenuated. The number of votes can be used in determining whether a portion of data samples should be masked, filtered, or attenuated.

Exemplary Implementation for a Heart Rate Monitoring Method

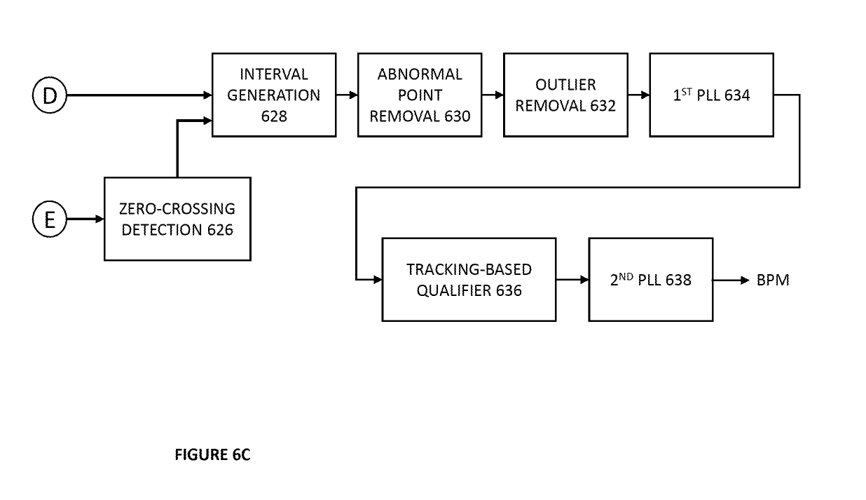

FIGS. 6A-C illustrate an exemplary implementation of a method for tracking a slow varying frequency present in one or more input signals provided by one or more sensors in a noisy environment, according to some embodiments of the disclosure. This exemplary implementation provide an example of how the various qualifiers shown in FIG. 5 can be combined, and how the data samples (once conditioned) can be conditioned and subsequently provided to a tracker to track the slow moving frequency.

Referring to FIG. 6A, it can be seen that a collection of qualifiers can be used to generate a plurality of selection signal “SEL” to control signal conditioning. The selection signals can be combined in any suitable manner, such as using “COMBINE” component 602 (in FIG. 6A) and “COMBINE” component 604 (in FIG. 6B). The “COMBINE” component 602 can include any one or more of the following: an AND operator, OR operator, a voting mechanism, a weighted voting mechanism, a decision tree, an artificial intelligence algorithm for classifying whether the data samples are good or bad, any suitable combination mechanisms for combining outputs from the qualifiers, etc. Other configuration of combine components can be used, and the manner and topology in which the selection signals are combined can also vary depending on the application. The selection signal can be a binary value of “0” and “1” or it can take on other values like a score. The selection signal can be associated with a particular data sample in a stream of data samples of the input signal to indicate whether the data sample should be used for tracking. The purpose of the qualifiers is to glean from one or more input signals whether signal conditioning is needed to improve the data samples being provided by the tracker. Another purpose of the qualifiers is to inform the tracker how to best process the data samples for optimal results. Broadly speaking, the selection signals can be used to control signal conditioning, and/or the tracker.

The implementation can include “FREQUENCY ANALYSIS FOR DETERMINING OVERLAP” part 606 for performing frequency analysis on the accelerometer data and the optical sensor data. “FREQUENCY ANALYSIS FOR DETERMINING OVERLAP” part 606 corresponds to “ACC. FREQ. OVERLAPS WITH LED FREQ.?” qualifier 510 of FIG. 5. “FREQUENCY ANALYSIS FOR DETERMINING OVERLAP” part 606 can generate a selection signal if the frequency information in the accelerometer data has common/overlapping components with the frequency information in the optical sensor data. The selection signal can cause certain data samples to be masked.

The implementation can include an “ABNORMAL POINT REMOVAL” part 608 for (identifying and) removing data samples which have abnormal values. The “ABNORMAL POINT REMOVAL” part 608 corresponds to “ABNORMAL?” qualifier 512 OF FIG. 5. The “ABNORMAL POINT REMOVAL” part 608 can take data samples of the input signal (from the optical sensor) and filter, mask, or remove certain data samples deemed to be abnormal. In one instance, abnormal points can be replaced with values which are of normal magnitude. The output of the “ABNORMAL POINT REMOVAL” part 608 can include data samples to be provided to another part for further processing.

The implementation can include a “SATURATION REMOVAL” part 610 for identifying data samples which is associated with a saturation condition. The “SATURATION REMOVAL” part 610 corresponds “SATURATION?” qualifier 514 of FIG. 5. The “SATURATION REMOVAL” part 610 can determine whether the data samples is associated with a saturation condition, and output a selection signal to indicate such a condition. The selection signal can cause the data samples to be masked.

The implementation can include “JUMP REMOVAL” part 612 for (identifying and) removing data samples which exhibits an abrupt jump. The “JUMP REMOVAL” part 612 corresponds to “JUMP?” qualifier 515 of FIG. 5. In this implementation, the “JUMP REMOVAL” part 612 takes the output from “ABNORMAL POINT REMOVAL” part 608 to further process the data samples produce data samples with data points having a shifted offset filtered, removed, or attenuated.

The implementation can include “1^{ST} FILTER (BANDPASS)” part 614 to process the data samples and attenuate signal content outside a reasonable frequency band of interest. The “1^{ST} FILTER (BANDPASS)” part 614 corresponds to “WITHIN NORMAL HEARTRATE FREQUENCY?” qualifier 508 of FIG. 5. A bandpass filter can be used to filter out frequencies which are not likely to be associated with a heartbeat, and in this implementation, the filter is applied to the output of “JUMP REMOVAL” part 612.

The implementation can include “BIG/SMALL REMOVAL” part 616 to remove data samples which has values that are too big or too small. The “BIG/SMALL REMOVAL” part 616 corresponds to “TOO BIG OR TOO SMALL” qualifier 516. In this implementation the “BIG/SMALL REMOVAL” part 616 takes the output from the “1^{ST} FILTER (BANDPASS)” part 614 and removes data samples which are too big or too small by generating a selection signal to cause those data samples to be filtered, masked, or attenuated.

The collection of parts seen in FIG. 6A performs a mixture of qualification and signal conditioning to process the optical sensor data (i.e., data samples of the input signal) to prepare the data samples for tracking. For instance, the implementation combines several selection signals (output of part 606, part 610, and part 616) to generate an aggregate selection signal. Referring to FIG. 6B, the aggregate selection signal can be provided to a MASK 618, which takes the aggregate selection signal and the output from “1^{ST} FILTER (BANDPASS)” as inputs. MASK 618 can then selectively mask, attenuate, or remove certain data samples (i.e., output from “1^{ST} FILTER (BANDPASS)” part 614) based on the aggregate selection signal. Effectively, MASK 618 can produce an output with certain undesirable portions of the data samples removed or attenuated. In some embodiments, MASK 618 can be replaced with a signal conditioner having one or more of the following: filtering, attenuating, and masking functionalities.

As it can be seen in FIG. 6B, further qualification and signal condition can be performed to further improve the data samples for tracking. The output from MASK 618 is provided to a “2^{ND} FILTER (BANDPASS)” part 620 to again filter out undesirable signals outside of a reasonable frequency band of interest. This “2^{ND} FILTER (BANDPASS)” part 620 is provided to primarily take the output from MASK 618 and remove the effect of MASK 618 has on the data samples, as masking can cause certain high frequency artifacts in the frequency information of the data samples.

The implementation can include “FFT FOR DETERMINING CLEAR PEAKS/SMOOTHNESS” part 622 to perform frequency domain analysis of the signal which is within the reasonable frequency band of interest. The “FFT FOR DETERMINING CLEAR PEAKS/SMOOTHNESS” part 622 corresponds to two qualifiers: “CLEAR PEAK?” qualifier 522 and “SLOW VARYING?” qualifier 506 of FIG. 5. In some embodiments, the FFT (“Fast Fourier Transform”) is performed on a sliding window of data samples, and multiple FFTs are performed for overlapping or non-overlapping sliding windows of data samples. By performing numerous FFTs on the sliding window of data samples which were samples of an input signal in time domain, the series of FFTs generated from the sliding window becomes a time-frequency representation of the input signal. The time-frequency representation comprises time-series frequency information of the input signal, where the series of frequency information generated from the sliding windows can each be associated with a particular data sample or a point in time. Other transforms can be used besides FFT, depending on the application, to transform a sliding window of data samples to the frequency domain. The sliding window provides a way to obtain a frequency information associated with instantaneous data samples, and each sliding window of which a particular data sample is a member can make a contribution to whether the particular data sample is associated with an FFT with clear peaks. The contributions from the sliding windows can be combined to provide a score for the particular data sample, and a threshold can be used to determine whether the score is high enough to keep the data sample for tracking.

If there is a clear peak, a further component of analysis is performed in the “FFT FOR DETERMINING CLEAR PEAKS/SMOOTHNESS” part 622 by comparing the frequencies for the peaks (i.e., prominent component with a clear peak) over time to determine whether there is a slow varying frequency over time. This component of analysis corresponds to the “SLOW VARYING?” qualifier 506 of FIG. 5. The “FFT FOR DETERMINING CLEAR PEAKS/SMOOTHNESS” part 622 can generate a selection signal to indicate that a slow varying frequency (i.e., a heartbeat) is present or not present. The selection signal from the “FFT FOR DETERMINING CLEAR PEAKS/SMOOTHNESS” part 622 can be combined, using “COMBINE” component 604 with the aggregate selection signal from “COMBINE” component 618 to generate a further aggregate selection signal. The further aggregate selection signal is provided to MASK 624 to mask or attenuate certain selected portions of data samples generated at the output of “1^{ST} FILTER (BANDPASS)” part 614 which are undesirable for tracking. The output of MASK 624 is a conditioned stream of data samples which is suitable for tracking, where the conditioned data samples is more likely to result in accurate tracking of the slow varying frequency than the original stream of data samples from the optical sensor. In some embodiments, MASK 624 can be replaced with a signal conditioner having one or more of the following: filtering, attenuating, and masking functionalities.

Referring to FIG. 6C, the output of MASK 624 is provided to a “ZERO-CROSSING DETECTION” part 626 to identify zero-crossings based on the conditioned stream of data samples. For instance, the part 626 can check whether the values have changed from a positive value to a negative value, or from a negative value to a positive value. It can be implemented to determine other types of crossings at a different threshold value besides zero. The purpose of “ZERO-CROSSING DETECTION” part 626 is to obtain information on oscillations or pulses present in the data samples. The processing of data samples to track the slow frequency can further include an “INTERVAL GENERATION” part 628 for determining interval information based on zero-crossing information of the data samples. For instance, adjacent pulses are used to compute the interval information between the adjacent pulses. The output of “INTERVAL GENERATION” part 628 can disregard certain intervals when the data samples had been masked (i.e., the part 628 can disregard certain interval information based on the aggregate selection signal from “COMBINE” component 604). This is because the interval information generated during periods where the data samples had been masked is not an accurate representation of intervals which could be used to track the heartbeat.

In some cases, conditioning can be performed on the output of “INTERVAL GENERATION” part 628, by providing “ABNORMAL POINT REMOVAL” part 630 and “OUTLIER REMOVAL” part 632. The “ABNORMAL POINT REMOVAL” part 630 can remove certain interval information if the interval information is deemed to be abnormally large or small, i.e., interval information which does not appear to be a heartbeat having a frequency of 0.5-3.5 Hertz. The “OUTLIER REMOVAL” part 632 can remove certain interval information if the interval information is indicates a large abrupt change in the interval information. A heart rate is assumed to be slow varying, and thus the interval information should not change drastically based on past interval information. An average can be calculated based on past and/or future interval information to determine whether the instantaneous interval information is an outlier. Outliers can removed to improve tracking. In some cases, outliers can be replaced with a more reasonable value, e.g., the average of previous and/or subsequent interval information.

The interval information is provided to a first phase locked loop (“1^{ST} PLL” 634) to track the slow varying frequency based on the frequency information suggested by the interval information (frequency is 1/interval). The interval information computes the amount of time between the zero-crossings, and serves as a nominal frequency to which the first phase locked loop can track. The first phase locked loop can track closely to the nominal frequency while following a slow varying frequency present in the input interval information.

The “1^{ST} PLL” 634 can further serve as a qualifier (besides being a tracker) if the output of the “1^{ST} PLL” 634 has some abnormal output values. The “TRACKING-BASED QUALIFIER” 636 can then be used to remove or attenuate abnormal output values generated by the “1^{ST} PLL” 634 prior to providing output values of the first phase locked loop as input to a second phase locked loop (“2^{ND} PLL” 638) to continue tracking the slow varying frequency. The output of the “2^{ND} PLL” 638 is the extracted frequency of the slow varying frequency in the input signal. The extracted frequency, if the slow varying frequency is a heart rate, can be provided in beats per minute (BPM).

In some embodiments, the tracking in “1^{ST} PLL” 634 and/or the “2^{ND} PLL” 638 can be improved by one or more qualifiers. For example, if a portion of Y number of samples (in time domain) has a prominent frequency component (e.g., 0.8 Hz) as determined by a qualifier which examines the frequency information of data samples), the portion of Y number of samples can be provided to a bandpass filter with passband surrounding the prominent frequency component (e.g., 0.5 Hz and 1.2 Hz) to further clean the signal and rid of frequency components which do not contribute to tracking the slow varying frequency. Such filtered data is much cleaner for the PLL to find accurate zero-crossing points when tracking the slow varying frequency.

Alternative or Complementary Tracking Method

A phase locked loop can be easy to implement but other methods are possible for tracking a slow varying frequency in a noisy environment. For instance, the time-frequency representation of the data samples can be used to generate a two dimensional space of values. Using highly overlapping windows of data samples, the method can generate frequency information for the overlapping windows. The frequency information for each of the overlapping windows can be, e.g., plotted with horizontal axis being frequency bins and vertical axis being the time slice of the window. An ideal input signal having a heartbeat would appear as a continuous contour running vertically in the spectrogram image. Such time-frequency representation can provide a basis for following a contour that can be formed by peaks in the frequency information changing in frequency slowly over time (i.e., running vertically in the spectrogram image). Peaks can be detected and followed over time using a contour tracking method to identify and track a slow varying frequency present in the data samples. In some embodiments, the method for tracking the slow varying frequency includes generating a time-frequency representation of the input signal based on the data samples, and tracking one or more contours present in the time-frequency representation to track the slow varying frequency.

In some embodiments, any suitable alternative or complementary tracking method can be improved by one or more qualifiers. For example, if a portion of Y number of samples (in time domain) has a prominent frequency component (e.g., 0.8 Hz) as determined by a qualifier which examines the frequency information of data samples), the portion of Y number of samples can be provided to a bandpass filter with passband surrounding the prominent frequency component (e.g., 0.5 Hz and 1.2 Hz) to further clean the signal and rid of frequency components which do not contribute to tracking the slow varying frequency. Such filtered data is much cleaner for the method when tracking the slow varying frequency.

Exemplary Results

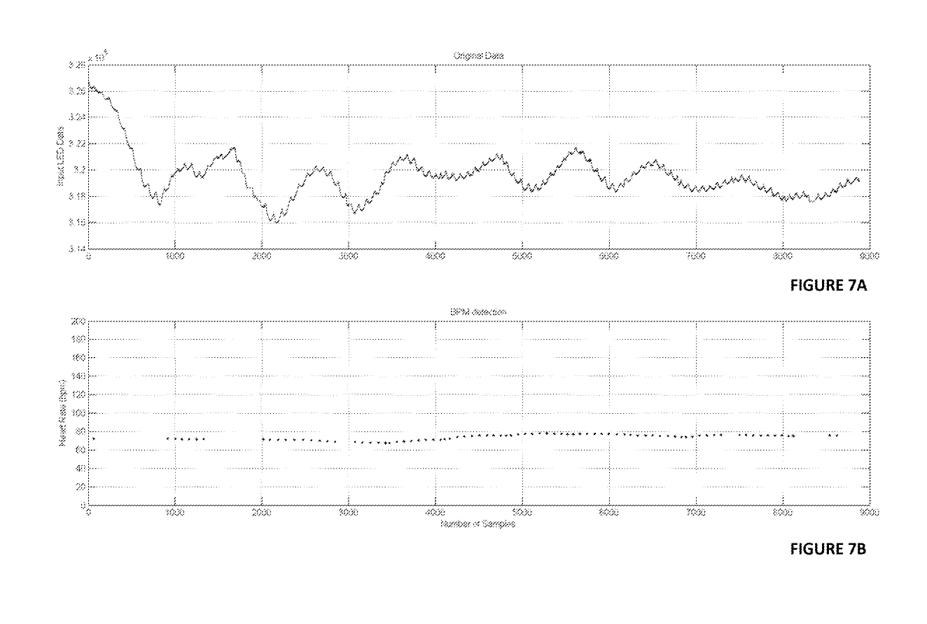

FIGS. 7A-B show a relatively good data set with minor noise and exemplary results by applying the improved method for tracking a slow varying frequency on such a data set, respectively, according to some embodiments of the disclosure. It can be seen that the improved method can generate a heart rate output accurately for a data set with minor noise. The heartbeat output appears smooth and slow varying.

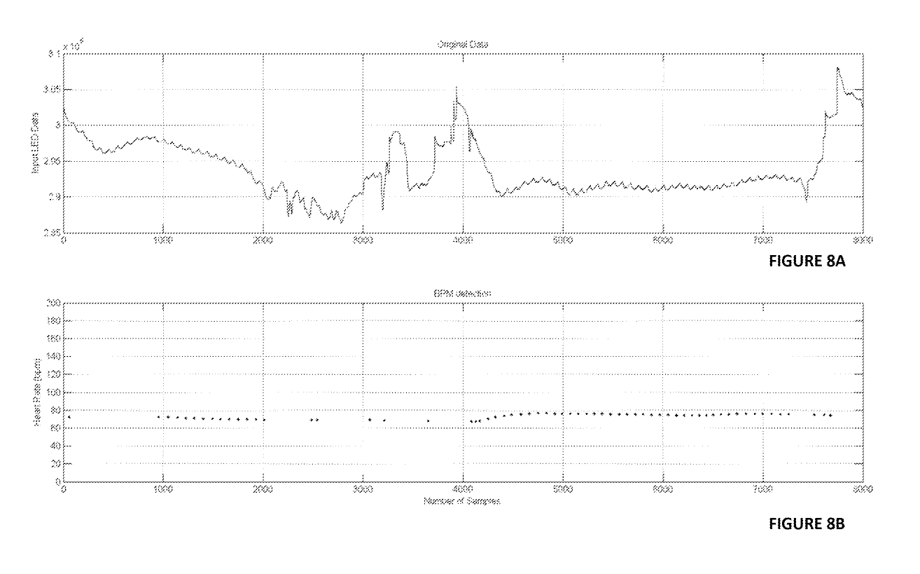

FIGS. 8A-B show a data set with a large noisy portion and exemplary results by applying the improved method for tracking a slow varying frequency on such a data set, respectively, according to some embodiments of the disclosure. Although there is a large amount of noise for samples 2000-4400, the improved method is still able to extract a heart rate during a noisy period.

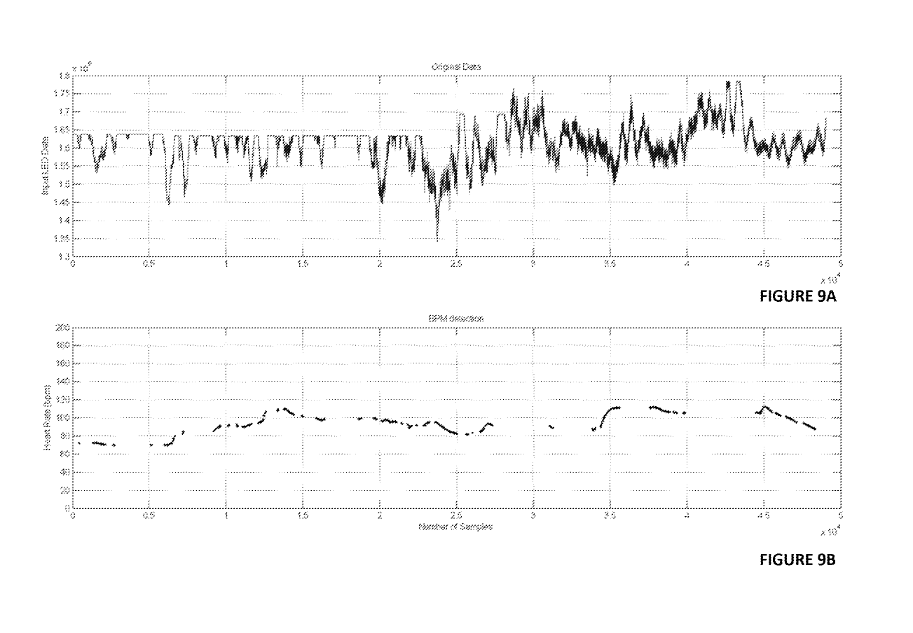

FIGS. 9A-B show a data set with a portion of data exhibiting saturation and a large amount of noise and exemplary results by applying the improved method for tracking a slow varying frequency on such a data set, respectively, according to some embodiments of the disclosure. Although there is a larger amount of distortion and noise (particularly in the first half of the data set), the method is able to track and produce a heart rate.

Variations and Implementations

FIGS. 6A-C can vary significantly to achieve equivalent or similar results, and thus should not be construed as the only possible implementation which leverages the qualifiers disclosed herein. Furthermore, qualifiers related to other sensors (“DIFFERENT SIGNAL QUALITIES” qualifier 518 and “COMMON COMPONENTS?” qualifier 520) can be readily added to the implementation shown in FIGS. 6A-C.

Many features of the present disclosure involves transforming data samples of the input signal into the frequency domain to perform spectral analysis of the data samples. These features may not necessarily have to be implemented using FFTs, although in some applications, the speed, availability, and lower complexity of FFTs can be desirable. Other possible transforms usable for extracting frequency information of the input signal can include but are not limited to one or more of the following exemplary transforms: wavelet transforms, Hartley transforms, cosine transforms, sine transforms, and so forth.

It is envisioned that the heart rate monitoring apparatus can be provided in many areas including medical equipment, security monitoring, patient monitoring, healthcare equipment, medical equipment, automotive equipment, aerospace equipment, consumer electronics, and sports equipment, etc.

In some cases, the heart rate monitoring apparatus can be used in professional medical equipment in a healthcare setting such as doctor's offices, emergency rooms, hospitals, etc. In some cases, the heart rate monitoring apparatus can be used in less formal settings, such as schools, gyms, homes, offices, outdoors, under water, etc. The heart rate monitoring apparatus can be provided in a consumer healthcare product.

The heart rate monitoring apparatus or parts thereof can take many different forms. Examples include watches, rings, wristbands, chest straps, headbands, headphones, ear buds, clamps, clips, clothing, bags, shoes, glasses, googles, hats, suits, necklace, attachments/patches/strips/pads which can adhere to a living being, accessories, portable devices, and so on. In particular, wearables technology (or referred often as “wearables”, i.e., electronics which are intended to be worn by humans or other living beings) can greatly leverage the benefits of the heart rate monitoring apparatus disclosed herein due to the wearables' portability and the heart rate monitoring technique's robustness against motion artifacts. Even in the presence of noise, the wearable can effectively track a heart rate. Besides wearables, portable or mobile devices such as mobile phones and tablets can also include a processor having the tracking functions, an analog front end, a light source and a light sensor (or an extension (wired or wireless) having the light source and light sensor) to provide a heart rate monitoring apparatus. Users can advantageously use a ubiquitous mobile phone to make a heart rate measurement. Furthermore, it is envisioned that the heart rate monitoring apparatus can be used in wired or wireless accessories such as cuffs, clips, straps, bands, probes, etc., to sense physiological parameters of a living being. These accessories can be connected to a machine configured to provide the processor and the analog front end. The analog front end could be provided in the accessory or in the machine.

Besides tracking a heart rate, the heart rate monitoring apparatus can be provided to sense or measure other physiological parameters such as oxygen saturation (SpO2), blood pressure, respiratory rate, activity or movement, etc. Besides humans, the heart rate monitoring apparatus can be provided to measure slow tracking frequencies present in signals sensing other living beings such as animals, insects, plants, fungi, etc.

In the discussions of the embodiments above, the capacitors, clocks, DFFs, dividers, inductors, resistors, amplifiers, switches, digital core, transistors, and/or other components can readily be replaced, substituted, or otherwise modified in order to accommodate particular circuitry needs. Moreover, it should be noted that the use of complementary electronic devices, hardware, software, etc. offer an equally viable option for implementing the teachings of the present disclosure. For instance, instead of processing the signals in the digital domain, it is possible to provide equivalent electronics that can process the signals in the analog domain.

In one example embodiment, any number of electrical circuits of the FIGURES may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), computer-readable non-transitory memory elements, etc. can be suitably coupled to the board based on particular configuration needs, processing demands, computer designs, etc. Other components such as external storage, additional sensors, controllers for audio/video display, and peripheral devices may be attached to the board as plug-in cards, via cables, or integrated into the board itself. In various embodiments, the functionalities described herein may be implemented in emulation form as software or firmware running within one or more configurable (e.g., programmable) elements arranged in a structure that supports these functions. The software or firmware providing the emulation may be provided on non-transitory computer-readable storage medium comprising instructions to allow a processor to carry out those functionalities. In some cases, application specific hardware can be provided with or in the processor to carry out those functionalities.

In another example embodiment, the electrical circuits of the FIGURES may be implemented as stand-alone modules (e.g., a device with associated components and circuitry configured to perform a specific application or function) or implemented as plug-in modules into application specific hardware of electronic devices. Note that particular embodiments of the present disclosure may be readily included in a system on chip (SOC) package, either in part, or in whole. An SOC represents an IC that integrates components of a computer or other electronic system into a single chip. It may contain digital, analog, mixed-signal, and often radio frequency functions: all of which may be provided on a single chip substrate. Other embodiments may include a multi-chip-module (MCM), with a plurality of separate ICs located within a single electronic package and configured to interact closely with each other through the electronic package. In various other embodiments, the slow varying frequency tracking functionalities may be implemented in one or more silicon cores in Application Specific Integrated Circuits (ASICs), Field Programmable Gate Arrays (FPGAs), and other semiconductor chips.

Note that the activities discussed above with reference to the FIGURES are applicable to any integrated circuits that involve signal processing, particularly those that can execute specialized software programs, or algorithms, some of which may be associated with processing digitized real-time data to track a slow moving frequency. Certain embodiments can relate to multi-DSP signal processing, floating point processing, signal/control processing, fixed-function processing, microcontroller applications, etc. In certain contexts, the features discussed herein can be applicable to medical systems, scientific instrumentation, wireless and wired communications, radar, industrial process control, audio and video equipment, current sensing, instrumentation (which can be highly precise), and other digital-processing-based systems. Moreover, certain embodiments discussed above can be provisioned in digital signal processing technologies for medical imaging, patient monitoring, medical instrumentation, and home healthcare. This could include pulmonary monitors, heart rate monitors, pacemakers, etc. Other applications can involve automotive technologies for safety systems (e.g., stability control systems, driver assistance systems, braking systems, infotainment and interior applications of any kind). Furthermore, powertrain systems (for example, in hybrid and electric vehicles) can use high-precision data conversion products in battery monitoring, control systems, reporting controls, maintenance activities, etc. It is envisioned that these applications can also utilize the disclosed improved method for tracking a slow moving frequency (e.g., tracking systems which are dampened to move at a frequency that changes slowly). In yet other example scenarios, the teachings of the present disclosure can be applicable in the industrial markets that include process control systems aiming to track a slow moving frequency to help drive productivity, energy efficiency, and reliability.

Note that with the numerous examples provided herein, interaction may be described in terms of two, three, four, or more parts. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of this Specification. In certain cases, it may be easier to describe one or more of the functionalities of a given set of flows by only referencing a limited number of electrical elements. It should be appreciated that the features of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

Note that in this Specification, references to various features (e.g., elements, structures, modules, components, steps, operations, parts, characteristics, etc.) included in “one embodiment”, “example embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments.

It is also important to note that the functions related to tracking a slow varying frequency, illustrate only some of the possible tracking functions that may be executed by, or within, systems illustrated in the FIGURES. Some of these operations may be deleted or removed where appropriate, or these operations may be modified or changed considerably without departing from the scope of the present disclosure. In addition, the timing of these operations may be altered considerably. The preceding operational flows have been offered for purposes of example and discussion. Substantial flexibility is provided by embodiments described herein in that any suitable arrangements, chronologies, configurations, and timing mechanisms may be provided without departing from the teachings of the present disclosure. Note that all optional features of the apparatus described above may also be implemented with respect to the method or process described herein and specifics in the examples may be used anywhere in one or more embodiments.

The ‘means for’ in these instances (above) can include (but is not limited to) using any suitable component discussed herein, along with any suitable software, circuitry, hub, computer code, logic, algorithms, hardware, controller, interface, link, bus, communication pathway, etc. In a second example, the system includes memory that further comprises machine-readable instructions that when executed cause the system to perform any of the activities discussed above.

What is claimed is:

1. A method for tracking a slow varying frequency present in one or more input signals provided by one or more sensors in a noisy environment, the method comprising:
receiving data samples of a first input signal;
extracting time-series frequency information of the first input signal based on the data samples of the first input signal;
determining whether the frequency information exhibits discontinuities in the time-series; and
processing the data samples to track the slow varying frequency based on whether the frequency information exhibits discontinuities.

2. The method of claim 1, wherein the slow varying frequency is representative of a heartbeat.

3. The method of claim 1, wherein the one or more sensors include one or more of the following: optical sensor, audio sensor, capacitive sensor, magnetic sensor, chemical sensor, humidity sensor, moisture sensor, pressure sensor, and biosensor.

4. The method of claim 1, wherein:
extracting time-series frequency information of the first input signal comprises determining frequency information of a first window of data samples and frequency information of a second window of data samples; and
determining whether the frequency information exhibits discontinuities in the time-series comprises determining whether a difference between the frequency information of the first window of data samples and frequency information of the second window of data samples is greater than a threshold.

5. The method of claim 4, further comprising:
applying a filter or mask to or removing a portion of the data samples which is associated with a discontinuity in the frequency information prior to processing the data samples to track the slow varying frequency.

6. The method of claim 1, further comprising:
processing the data samples with a filter to substantially attenuate signal content outside of a reasonable frequency band of interest corresponding to the slow varying frequency of the input signal before extracting time-series frequency information of the input signal.

7. The method of claim 6, wherein:
the filter is a low-pass filter or a band-pass filter; and
the reasonable frequency band of interest comprises frequencies between 0.5 Hertz to 3.5 Hertz.

8. The method of claim 1, further comprising:
receiving data samples of a signal indicative of motion of the one or more sensors;
extracting time-series motion frequency information based on the data samples of the signal indicative of motion; and
if the motion frequency information corresponding to a particular time has one or more components common with frequency information corresponding to the same particular time, applying a filter or mask to or removing a portion of the data samples associated with the same particular time prior to processing the data samples to track the slow varying frequency.

9. The method of claim 1, further comprising:
applying a filter or mask to or removing a portion of the data samples indicative of a saturation condition of the one or more sensors prior to processing the data samples to track the slow varying frequency.

10. The method of claim 1, further comprising:
applying a filter or mask to or removing a portion of the data samples which is outside of an expected range of values prior to processing the data samples to track the slow varying frequency.

11. The method of claim 1, further comprising:
applying a filter or mask to a portion of the data samples which are associated with an offset exceeding a predetermined threshold prior to processing the data samples to track the slow varying frequency.

12. The method of claim 1, further comprising:
receiving data samples of a second input signal, wherein the first input signal is provided by a first optical sensor has a first signal quality that is different, in presence of motion or source of noise, from a second signal quality of the second input signal provided by a second optical sensor;
determining whether signal quality of the first input signal and signal quality of the second input signal are substantially different for a particular time; and
processing the data samples to track the slow varying frequency based on whether signal quality of the first input signal and signal quality of the second input signal are substantially different for a particular time.

13. The method of claim 1, further comprising:
receiving data samples of a second input signal, wherein the first input signal is provided by a first optical sensor for sensing a first bandwidth and the second input signal is provided by a second optical sensor for sensing a second bandwidth different from the first bandwidth;
extracting time-series frequency information based on the data samples of the second input signal; and
if the frequency information of the first input signal corresponding to a particular time has one or more components common with frequency information of the second input signal corresponding to the same particular time, applying a filter or mask to or removing a portion of the data samples associated with the same particular time prior to processing the data samples to track the slow varying frequency.

14. The method of claim 1, wherein processing the data samples to track the slow varying frequency comprises:
determining interval information based on zero-crossing information of the data samples; and
providing the interval information to a first phase locked loop to track the slow varying frequency.

15. The method of claim 14, wherein processing the data samples to track the slow varying frequency further comprises:
removing abnormal output values generated by the first phase-locked loop prior to providing output values of the first phase locked loop as input to a second phase locked loop to track the slow varying frequency.

16. The method of claim 1, wherein processing the data samples to track the slow varying frequency comprises:
generating a time-frequency representation of the input signal based on the data samples; and
tracking one or more contours present in the time-frequency representation to track the slow varying frequency.

17. An apparatus for tracking a slow varying frequency present in one or more input signals provided by one or more sensors in a noisy environment, the apparatus comprising the following parts which can be provided on a processor or circuit:
a qualifier to:
receive data samples of a first input signal;
extract time-series frequency information of the first input signal based on the data samples of the first input signal;
determine whether the frequency information exhibits discontinuities in the time-series; and
a tracker to process the data samples to track the slow varying frequency based on whether the frequency information exhibits discontinuities.

18. The apparatus of claim 17, further comprising:
a signal conditioner to apply a mask to or removing a portion of the data samples which is associated with a discontinuity in the frequency information prior to processing the data samples to track the slow varying frequency.

19. A non-transitory computer-readable medium comprising one or more instructions, said instructions for tracking a slow varying frequency present in one or more input signals provided by one or more sensors in a noisy environment, that when executed on a processor configure the processor to:
receiving data samples of a first input signal;
extracting time-series frequency information of the first input signal based on the data samples of the first input signal;
determining whether the frequency information exhibits discontinuities in the time-series; and
processing the data samples to track the slow varying frequency based on whether the frequency information exhibits discontinuities.

20. The non-transitory computer-readable medium of claim 19, further comprising:
applying a filter or mask to or removing a portion of the data samples which is associated with a discontinuity in the frequency information prior to processing the data samples to track the slow varying frequency.
